(19) United States
(12) Patent Application Publication
(10) Pub. No.: US 2025/0367352 A1
(43) Pub. Date: Dec. 4, 2025

(54) BREAST PUMP AND BREAST PUMP VALVE ASSEMBLY

(71) Applicant: Mayborn (UK) Limited, Newcastle Upon Tyne (GB)

(72) Inventors: Matthew James Gossington, Newcastle Upon Tyne (GB); Robert Kenneth Alexander Jervis, Newcastle Upon Tyne (GB)

(21) Appl. No.: 18/876,414

(22) PCT Filed: Jun. 14, 2023
(86) PCT No.: PCT/GB2023/051552

§ 371 Date: Dec. 18, 2024

(30) Foreign Application Priority Data
Jun. 20, 2022 (GB) 2209039.3

(51) Int. Cl. A61M 1/06

(52) U.S. Cl. CPC A61M 1/062

(57) ABSTRACT

A breast pump valve assembly includes a first chamber and a second chamber between first and second one-way valves. The first valve allows fluid into the second chamber and the second valve allows out of the second chamber. A flow path between the second chamber and an ambient atmosphere has a third valve. A displacement member separates the first chamber and the second chamber. Negative pressure to the first chamber causes the displacement member to increase the volume of the second chamber and draw fluid into the second chamber through the first valve. When pressure in the first chamber is equal to or greater than pressure in the second chamber, the pressure in the first chamber causes the displacement member to decrease the volume of the second chamber. The third valve can be opened so movement of the displacement member directs fluid from the second chamber through the third valve.

### Introduction

The present invention relates to a breast pump, in particular a breast pump and a breast pump valve assembly each for use in expressing milk.

Breast pumps, commonly used by mothers to express breast milk, generally include a breast receiving part, also known as a breast shield or horn, for receiving the breast and nipple, and a pump to generate and apply suction. The breast receiving part is shaped so that a surface can contact the breast and form an air-tight seal against the skin to ensure suction is applied to the nipple within the breast receiving part.

Suction, also known as vacuum pressure, vacuum or negative pressure, is provided by a negative pressure source, such as a manual or electrical pump. Manual breast pumps require the user to manually actuate a membrane or piston using a handle mounted to or on the breast receiving part. Electric breast pumps use an electrically-powered pump unit which is either mounted to the breast receiving part, or is remote therefrom and is fluidly connected to the breast receiving part by vacuum tubing and connectors.

Suction generated by both manual and electrical pumps is provided in pulses causing suction to be applied to the nipple in cycles. The suction stimulates expression of breastmilk from the nipple which flows away from the breast receiving part and is collected in a suitable container arranged in fluid communication.

A one-way valve is provided between the breast receiving part and the container to control fluid, typically breastmilk, flowing into the container while preventing suction from the pump being applied to the container. Within one suction cycle, the pump generates a suction pulse within the breast receiving part, before releasing or venting the suction to atmospheric pressure so that expressed breastmilk is able pass through the one-way valve to the container. Subsequent cycles repeat the pulse and release process. In manual pumps, the rate and intensity of each cycle is controlled by the user whereas electrical pumps are electronically controlled using either pre-programmed or manually adjusted settings.

A known problem of such breast pumps is that pumping is inefficient due to the release of suction after each cycle, which necessitates repeatedly generating suction from atmospheric pressure within the breast receiving part. A further drawback is that release of suction causes stimulation of the nipple to cease, slowing the overall rate of milk expression. A yet further drawback is that the release of suction can cause the air-tight seal between the breast receiving part and the breast to fail, leading to leaks and requiring the user to maintain the positioning of the breast pump against the breast by applying pressure, which causes discomfort.

Another problem of certain known breast pumps is that collection of expressed milk is slow. The pulsed suction provided by the pump to provide effective expression of breastmilk inhibits onward flow of the expressed milk into a collecting container. Thus, breastmilk accumulates within the breast pump assembly because it is not transferred to the container within a suction cycle. A yet further drawback is that the accumulated breastmilk can back up to the breast receiving portion, also leading to leaks and requiring the user to maintain the positioning of the breast pump against the breast by applying pressure, which causes discomfort.

A further problem of known breast pumps is that they require large pump devices able to pump large volumes of fluid. In particular, if a breast pump has a large residual volume between the breast receiving part and the container then a large volume of residual air must be pumped out to generate sufficient suction to initiate breastmilk expression. This requires the pump to be either a separate, remote pump or, if integrated with the breast receiving part, adds considerable weight and bulk to the breast receiving part.

It would therefore be useful to provide a breast pump with improved efficiency. In particular, it would be useful to provide a breast pump which provides efficient suction and which improves the rate of breastmilk expression. It would be useful to provide a breast pump which is convenient for a user to wear.

It would also be useful to provide a breast pump which allows efficient collection of expressed breastmilk. In particular, it would be useful to provide a breast pump which collects breastmilk efficiently while maintaining suction to a user's nipple.

It is a further object of the present invention to provide a breast pump with improved comfort and reduced leaks for the user.

### SUMMARY OF THE INVENTION

As used herein, the term ‘fluid’ refers to a gas, typically air, or a liquid, typically breastmilk, or a mixture of a gas and a liquid. Unless explicitly stated otherwise, a breast pump, a valve assembly, or any components or constituent parts thereof which are described as suitable to act upon a fluid are suitable to act upon a gas, a liquid, and a mixture of a gas and a liquid.

According to a first aspect of the invention, there is provided a breast pump valve assembly including:
a first chamber for fluid connection to a negative pressure source;
a second chamber defined between a first one-way valve and a second one-way valve, wherein the first one-way valve is configured to allow flow of fluid into the second chamber and the second one-way valve is configured to allow flow of fluid out of the second chamber,
a fluid flow path between the second chamber and an ambient atmosphere, the fluid flow path including a selectively openable third valve; and
a displacement member fluidly separating the first chamber and the second chamber, wherein the displacement member is configured such that application of negative pressure to the first chamber causes a first movement of the displacement member to thereby increase the volume of the second chamber and draw fluid into the second chamber through the first one-way valve,
wherein the displacement member is further configured such that, when pressure in the first chamber is equal to or greater than pressure in the second chamber, the pressure in the first chamber causes a second movement of the displacement member to thereby decrease the volume of the second chamber; and
wherein the third valve is configured to be selectively opened in response to the second movement of the displacement member, such that the second movement of the displacement member directs fluid from the second chamber through the third valve.

Aptly, the displacement member is configured such that the second movement of the displacement member further directs fluid from the second chamber through the second one-way valve.

Aptly, the third valve is further configured to be selectively openable in response to the first movement of the displacement member, thereby drawing fluid into the second chamber through the third valve.

Aptly, the displacement member is configured to move between a neutral position when the first chamber is at atmospheric pressure, and a displaced position when the first chamber is under negative pressure.

Aptly, in the neutral position, the displacement member substantially closes at least a portion of the second chamber.

Aptly, in the neutral position, the displacement member blocks the fluid flow path.

Aptly, the displacement member is integrally formed with one or more of: the first one-way valve, the second one-way valve, or the third valve.

Aptly, the displacement member includes a flexible diaphragm configured to deform upon application of negative pressure to the first chamber.

Aptly, at least one of the first and second one-way valves is a duck bill valve.

Aptly, the third valve is a slit valve, and preferably a domed cross-slit valve.

Aptly the breast pump valve assembly further includes a housing including a first housing member defining the second chamber, and a second housing member defining the first chamber. More aptly, the displacement member is positioned between the first housing member and the second housing member.

Aptly, the breast pump valve assembly further includes a fluid inlet, wherein the first one-way valve is configured to allow flow of fluid from the fluid inlet to the second chamber and, preferably, further including an inlet port for fluidly coupling the inlet to a breast receiving part of a breast pump.

Aptly, the breast pump valve assembly further includes a fluid outlet, wherein the second one-way valve is configured to allow flow of fluid from the second chamber to the outlet and. More aptly, valve assembly further includes an outlet port for fluidly coupling the outlet to a fluid collection container.

Aptly, the breast pump valve assembly is further configured such that, in response to the second movement of the displacement member, the fluid flow path directs air from the second chamber through the third valve.

Aptly, the fluid flow path is a recessed channel.

According to another aspect of the invention, there is provided a breast pump valve assembly including:
a variable volume second chamber defined between a first one-way valve and a second one-way valve, wherein the first one-way valve is configured to allow flow of fluid into the second chamber and the second one-way valve is configured to allow flow of fluid out of the second chamber,
a third valve is configured to provide a selectively openable fluid flow path between the second chamber and an ambient atmosphere; and
a displacement member configured for varying the volume of the second chamber, wherein the displacement member is configured such that a first movement of the displacement member to increase the volume of the second chamber thereby draws fluid into the second chamber through the first one-way valve;
wherein the third valve is configured to be selectively opened in response to a second movement of the displacement member, thereby directing fluid from the chamber through the third valve.

Aptly, the displacement member is further configured such that the second movement of the displacement member decreases the volume of the second chamber.

Aptly, the breast pump valve assembly further includes a first chamber, wherein the displacement member fluidly separates the first chamber and the second chamber.

Aptly, the breast pump valve assembly further includes a vacuum port for fluidly connecting the first chamber to a negative pressure source.

Aptly, the displacement member is configured such that application of negative pressure to the first chamber causes the first movement of the displacement member to thereby increase the volume of the second chamber and draw fluid through the first one-way valve into the second chamber.

Aptly, the displacement member is further configured such that, when pressure in the first chamber is equal to or greater than pressure in the second chamber, the pressure in the first chamber causes the second movement of the displacement member.

Aptly, the breast pump valve assembly further includes an actuator configured to operably move the displacement member to thereby vary the volume of the second chamber.

According to another aspect of the invention, there is provided a breast pump including:
a first chamber and a vacuum port for fluidly connecting the first chamber to a negative pressure source;
a breast receiving part, a fluid collection container, and a fluid flow path extending between the breast receiving part and the fluid collection container;
a second chamber defined between a first one-way valve and a second one-way valve in the fluid flow path, wherein the first one-way valve is configured to allow flow of fluid from the breast receiving part into the second chamber, and the second one-way valve is configured to allow flow of fluid from the second chamber to the fluid collection container,
a fluid flow path between the second chamber and an ambient atmosphere, the fluid flow path including a selectively openable third valve; and
a displacement member fluidly separating the first chamber and the second chamber, wherein the displacement member is configured such that application of negative pressure to the first chamber causes a first movement of the displacement member to thereby increase the volume of the second chamber and draw fluid from the breast receiving part through the first one-way valve into the second chamber wherein the displacement member is configured such that, increase of pressure in the first chamber towards atmospheric pressure causes a second movement of the displacement member to thereby decrease the volume of the second chamber; and
wherein the third valve is configured to be selectively openable in response to the second movement of the displacement member, thereby directing fluid from the second chamber through the third one-way valve.

Aptly, the displacement member is configured such that the second movement of the displacement member further directs fluid from the second chamber through the second one-way valve to the fluid collection container.

Aptly, the breast pump further includes a negative pressure source fluidly connected to the vacuum port.

It will be appreciated that any of the features described above in relation to the first aspect of the invention may apply equally to the second and third aspects of the invention.

Certain examples provide a breast pump valve assembly enabling improved breast pump efficiency compared to known breast pumps.

In particular, certain examples enable a breast pump with improved efficiency for expressing breastmilk from the nipple is provided because the breast pump is able to apply suction continuously to a user's nipple.

In particular, certain examples enable a breast pump with improved efficiency in collecting expressed breastmilk in a container. This is because the breast pump is able to expel or displace into a collecting container a substantial portion, or substantially all, of the breastmilk drawn into the breast pump valve assembly. More particularly, the breast pump is able to expel or displace into a collecting container a substantial portion, or substantially all, of the breastmilk drawn into the breast pump valve assembly during each suction cycle.

Certain examples provide a breast pump valve assembly providing improved user comfort compared to known breast pumps.

Certain examples provide a breast pump valve assembly which enable reduces leakage from a breast pump. In particular, certain examples provide a breast pump assembly which reduces leakage between the breast pump and a user's breast.

### BRIEF DESCRIPTION OF THE DRAWINGS

Embodiments of the invention are now described, by way of example only, with reference to the accompanying drawings, in which:

FIG. 1 shows a schematic of a first known breast pump valve assembly of the prior art;

FIG. 2 shows an exploded perspective view from below of the components of a breast pump valve assembly according to a known configuration of the schematic apparatus of FIG. 1;

FIG. 3 shows an exploded perspective view from above of the components of the known breast pump valve assembly of FIG. 2;

FIG. 4 shows a cross-sectional view of the known breast pump valve assembly of FIG. 2;

FIG. 5 shows a cross-sectional view of a breast pump including the known breast pump valve assembly of FIG. 2;

FIG. 6 shows a perspective view of a second known breast pump valve;

FIG. 7 shows a cut-away perspective views from (a) above and (b) below of the known breast pump valve of FIG. 6;

FIG. 8 shows a perspective view of known breast pump valve of FIG. 6 mounted to a breast receiving part;

FIG. 9 shows a third known example of a breast pump valve assembly including an alternative displacement member;

FIG. 10 shows an exploded view of a fourth known breast pump valve assembly;

FIG. 11 shows the lower member of the known breast pump valve assembly of FIG. 10;

FIG. 12 shows the underside of the upper member of the known breast pump valve assembly of FIG. 10;

FIG. 13 shows the underside of the support member of the known breast pump valve assembly of FIG. 10;

FIG. 14 shows an exploded perspective view from above of an example breast pump valve assembly according to the present invention;

FIG. 15 shows an exploded perspective view from below of the example breast pump valve assembly of FIG. 14; and

FIG. 16 shows a cross-sectional view of the example breast pump assembly of FIG. 14 in an assembled configuration.

In the drawings, like reference numerals refer to like parts.

### DETAILED DESCRIPTION

Certain terminology is used in the following description for convenience only and is not limiting. The words ‘lower’ and ‘upper’ designate directions in the drawings to which reference is made and are with respect to the described component when assembled and mounted. The words ‘inner’, ‘inwardly’ and ‘outer’, ‘outwardly’ refer to directions toward and away from, respectively, a designated centreline or a geometric centre of an element being described (e.g. central axis), the particular meaning being readily apparent from the context of the description.

Further, unless otherwise specified, the use of ordinal adjectives, such as, ‘first’, ‘second’, ‘third’ etc. merely indicate that different instances of like objects are being referred to and are not intended to imply that the objects so described must be in a given sequence, either temporally, spatially, in ranking or in any other manner.

### Breast Pump Valve Assemblies of the Prior Art

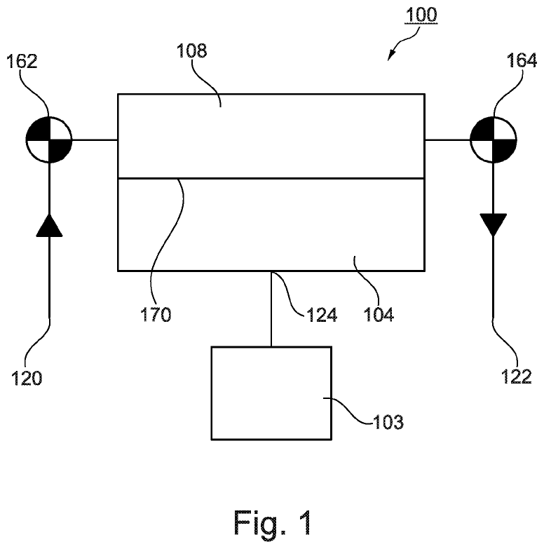

Referring now to FIG. 1, there is shown a schematic of a known breast pump valve assembly 100 including a suction chamber 104, for fluid connection to a negative pressure source 103, and a fluid chamber 108 defined between a first one-way valve 162 and a second one-way valve 164. The first one-way valve 162 is configured to allow fluid to flow into the fluid chamber 108. The second one-way valve 164 is configured to allow fluid to flow out of the fluid chamber 108.

The valve assembly 100 further includes a fluid inlet 120 and a fluid outlet 122. The first one-way valve 162 is positioned between the fluid inlet 120 and the fluid chamber 108 and configured to allow flow of fluid from the fluid inlet 120 into the fluid chamber 108.

The valve assembly 100 further includes a displacement member 170, arranged to fluidly separate the suction chamber 104 from the fluid chamber 108. The displacement member 170 is configured such that application of negative pressure to the suction chamber 104 causes the displacement member 170 to move and thereby increase the volume of fluid chamber 108, drawing fluid (e.g. breast milk) into the fluid chamber 108 through the first one-way valve 162.

As the pressure in the suction chamber 104 becomes equal to or greater than a pressure in the fluid chamber 108, the displacement member 170 moves to decrease the volume of the fluid chamber 108. As such, fluid in the fluid chamber 108 is directed out of the fluid chamber 108 through the second one-way valve 164, e.g. towards a fluid collection container (not shown).

The displacement member 170 is configured to assume a neutral position when the pressure in the suction chamber 104 is substantially equal or greater than the pressure in fluid chamber 108. For example, the displacement member 170 assumes the neutral position when the suction chamber 104 is substantially at atmospheric pressure. The displacement member 170 then moves to a displaced position when the suction chamber is under negative pressure. When the pressure in suction chamber 104 increases to equal or be greater than the pressure in the fluid chamber then the displacement member 170 attempts to assume, that is revert to, the neutral position.

The suction chamber 104 includes a vacuum port 124 configured for fluid connection to the negative pressure source 103. The vacuum port 124 is positioned such that the displacement member 170 moves towards the vacuum port 124 upon application of negative pressure to the suction chamber 104.

In this way, it is expected that when the pressure in suction chamber 104 increases to equal or be greater than the pressure in the fluid chamber then the displacement member 170 of the known valve assembly will revert to the neutral position. This is to ensure a substantial portion of or substantially all fluid is removed from the fluid chamber 108 via the second one-way valve 164.

However, the inventors of the present invention have discovered that, in practice, it is difficult to ensure that a substantial portion of, or substantially all, fluid is removed from the fluid chamber 108. The second one-way valve 164 is configured to allow flow of fluid from the fluid chamber 108 to the fluid outlet 122. In this example, the second one-way valve is positioned between the fluid chamber 108 and the fluid outlet 122.

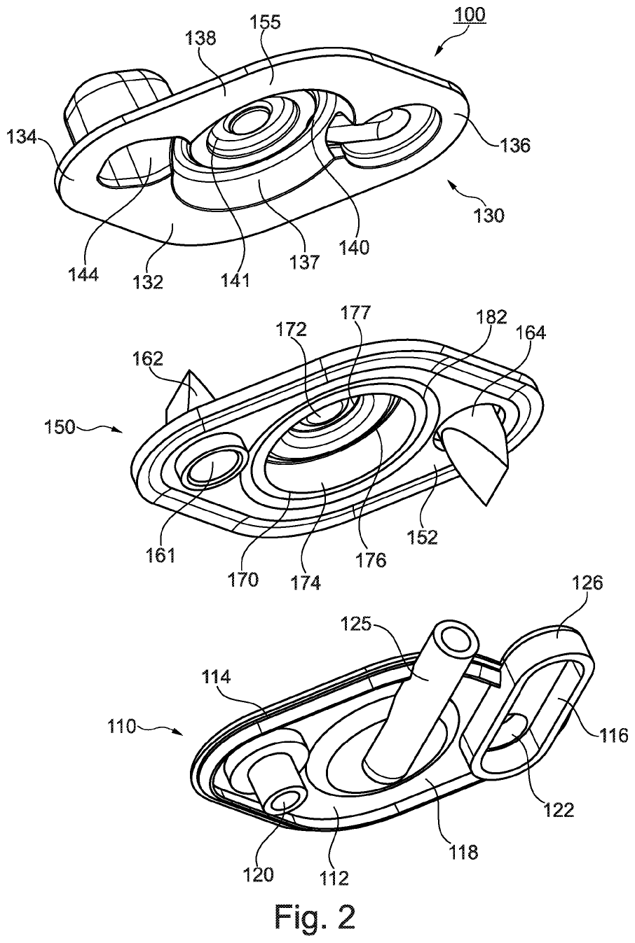

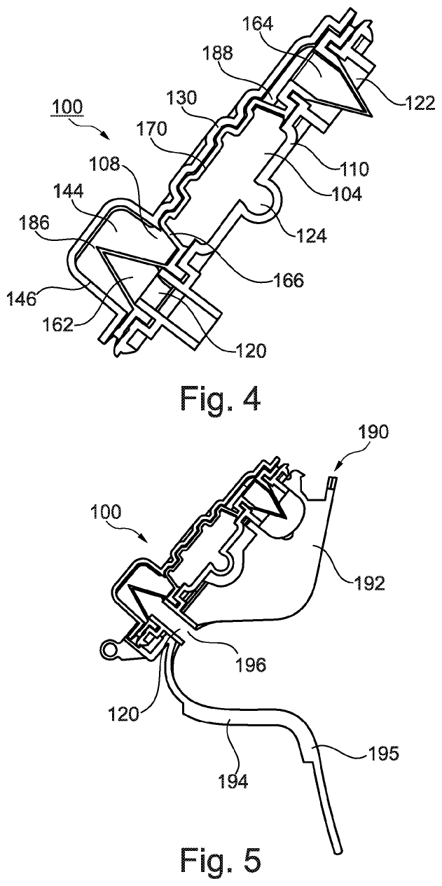

FIGS. 2 to 5 show a first known example configuration of the valve assembly 100 of FIG. 1. In this example, the valve assembly 100 includes a housing having a first housing member 130 and a second housing member 110. In this example, the first housing member is an upper member 130 and the second housing member is a lower member 110. The upper member 130 generally defines the fluid chamber 108. The lower member 110 generally defines the suction chamber 104. The displacement member 170 is positioned in between the upper member 130 and the lower member 110, thereby fluidly separating the suction chamber 104 and the fluid chamber 108.

In this example, first and second one-way valves 162, 164 and the displacement member 170 are arranged along a lengthways axis of a support member 150.

In this example, the lower member 110 includes a first opening, defining the fluid inlet 120, a second opening, defining the fluid outlet 122, and a third opening, defining the vacuum port 124.

In this example, the lower member 110 is provided as a plate having upper and lower (or inner and outer) surfaces 111, 112.

The fluid inlet 120, fluid outlet 122 and vacuum port are provided through the lower member 110 and arranged along a lengthways axis of the lower member 110.

The inlet 120 includes an inlet port for fluidly coupling the inlet to a breast receiving part of a breast pump.

The outlet 122 includes an outlet port for fluidly coupling the outlet to a fluid collection container and is configured for attachment to tubing or other fluid pathways extending to a fluid collection container.

The vacuum port 124 is configured for fluid connection to a vacuum source or source of negative pressure (e.g. a pump).

In this example, the upper member 130 is provided as a plate having upper and lower (or outer and inner) surfaces 131, 132. The cavity 137 defines at least a portion of the fluid chamber 108 between the first one-way valve 162 and second one-way valve 164.

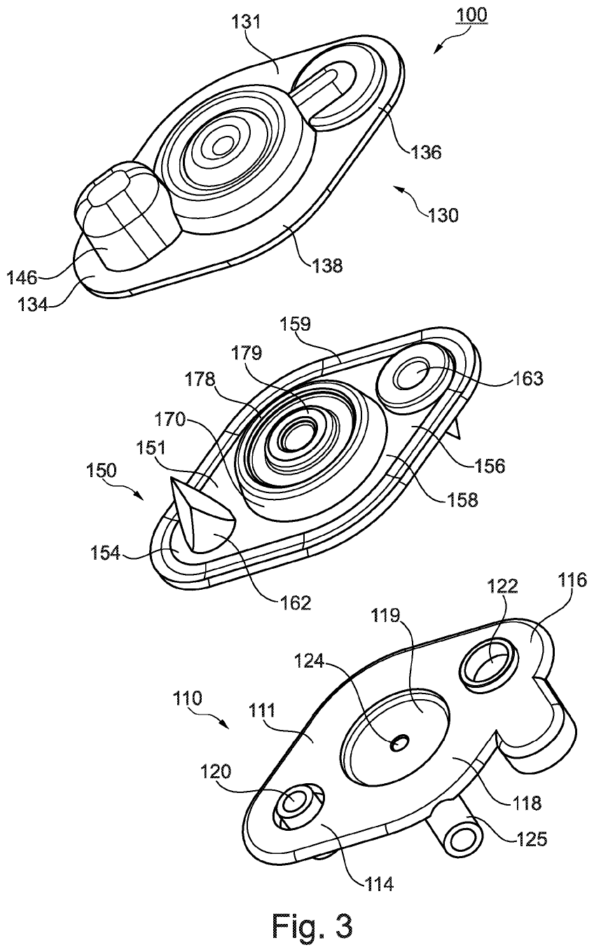

A recess 144 is provided in the lower surface 132 of the first end portion 134. The recess 144 is sized and shaped to house the first one-way valve 162 (as shown in FIG. 3).

In this example, the support member 150 is positioned between the upper member 130 and the lower member 110. The support member 150 includes the displacement member 170, the first one-way valve 162 and the second one-way valve 164. In this way, the displacement member 170, and first and second one-way valves 162, 164 are integrally formed.

The support member 150 includes a first surface 151 facing the upper member 130 and a second surface 152 facing the lower member. An edge portion 159 extends around the support member 150 which may be clamped between the inner surface 132 of the upper member 130 and the inner surface 111 of the lower member 110.

The support member 150 includes a first opening 161 configured to align with the fluid inlet 120. The first opening 161 is provided with the first one-way valve 162, adapted to selectively allow fluid to pass through the first opening 161.

In this example, the first one-way valve 162 is a duck bill valve including a pair of mutually-opposed inclined walls, extending from the first surface 151 of the displacement member 150, angled to meet at a first ridge parallel to and distal from the first surface 151.

A slit is provided along and through the first ridge, adapted to move between open and closed positions in response to pressure differences across the mutually-opposed inclined walls, as is known in the art. In this way, the first one-way valve 162 is configured to allow fluid to pass through the first opening 161 in a direction from the second surface 152 to the first surface 151, while preventing fluid passing through in a reverse direction.

The support member 150 includes a second opening 163 configured to align with the fluid outlet 122. The second opening 163 is provided with the second one-way valve 164, adapted to selectively allow fluid to pass through the second opening 163.

In this example, the second one-way valve 164 is a duck bill valve.

The support member 150 further includes the displacement member 170, which in this example is a flexible diaphragm. The flexible diaphragm 170 is configured to deform upon application of negative pressure to the suction chamber 104.

The diaphragm 170 is positioned such that it aligns with the recess 119 on the lower member 110 and the cavity 137 in the upper member 136. In this way, the diaphragm 170 is configured to fluidly separate the recess 119 (forming the suction chamber 104) and the cavity 137 (forming at least part of the fluid chamber 108).

The diaphragm 170 is shaped to correspond to the cavity 137 in the upper member 130. In this way, in the neutral position the diaphragm 170 is expected to abut against an inner surface of the cavity, thereby minimizing the volume of the fluid chamber 108 as much as possible and ensuring substantially all fluid can be expelled therefrom. However, in practice, it is difficult to ensure that a substantial portion of, or substantially all, fluid is displaced from the fluid chamber 108.

The diaphragm 170 is provided in the centre portion 158 of the support member 150. The diaphragm 170 may include a base 172, recessed from the second surface 152 by an angled wall 174 extending around the base 172.

Features of the support member 150 are configured to engage with corresponding features of the upper and lower members 110, 130.

The outer rim portion 159 and inner rim portion 182 of the support member 150 are shaped to be clamped between the upper and lower members 130, 110 when coupled to form the housing. When clamped, the support member 150 divides the housing to form the fluid chamber 108 between the support member 150 and the upper member 130 and the suction chamber 104 between the displacement member 170 of the support member 150 and the lower member 110.

The first one-way valve 162 caps the inlet 120 and selectively allows fluid into the housing from the fluid inlet 120.

The second one-way valve 164 caps the outlet 122 and selectively allows fluid to leave the housing through the fluid outlet 122.

The diaphragm 170 abuts the upper member 130 lower surface 132.

The fluid chamber 108 is provided by the volume defined between the upper member 130 and the first side 151 of the support member 150, delimited by the clamping of the outer rim portion 159 between the upper member 130 and lower member 110.

The diaphragm 170 is expected to remain in the neutral position as long as there are equal pressures in the fluid chamber 108 and suction chamber 104.

The diaphragm 170 is configured to respond to negative pressure provided to the suction chamber 104. In use, negative pressure may be introduced to the suction chamber 104 via the vacuum port 124, using any suitable negative pressure source, for example a manual or electric pump.

Within a first cycle, application of negative pressure within the suction chamber 104 deforms the diaphragm 170 to thereby decrease the volume of the suction chamber 104 and expand the volume of the fluid chamber 108.

As the volume of the fluid chamber 108 increases, the pressure in the fluid chamber 108 decreases, thereby creating a pressure differential across the first one-way valve 162. Due to the pressure differential, the first one-way valve 162 opens to draw fluid from the fluid inlet 120 into the fluid chamber 108.

As negative pressure increases, the diaphragm 170 further deforms, moving further away from the centre portion 138 of the upper housing 130. Negative pressure in the suction chamber 104 increases until the first cycle reaches a maximum negative pressure. At the maximum negative pressure, the diaphragm 170 is in a displaced position. In the displaced position, the volume of fluid chamber 108 is at a maximum and the volume of the suction chamber 104 is at a minimum.

To complete the first cycle, negative pressure in the suction chamber 104 is released, allowing the diaphragm 170 to attempt to return to its undeformed neutral shape. The diaphragm 170 therefore attempts to move back to its neutral position in which a portion of the fluid chamber 108 is substantially closed. Referring now to FIG. 5, there is shown a breast pump 190 suitable for use expressing breastmilk, including the valve assembly 100 of the example shown in FIGS. 2 to 4.

The breast pump 190 further includes a breast receiving part 192 (for example a breast horn), a fluid collection container (not shown) and a fluid flow path extending between the breast receiving part 192 and the fluid collection container. The fluid flow path extends through the valve assembly 100, with the first and second one-way valves 162, 164 disposed in the fluid flow path. The valve assembly 100 is configured substantially the same as the valve assembly 100 described above with the fluid chamber 108 defined between the first and second one-way valves 162, 164.

As such, the first one-way valve 162 is configured to allow flow of fluid (e.g. breast milk) from the breast receiving part 192 into the fluid chamber 108 and the second one-way valve 164 is configured to allow flow of fluid (e.g. breast milk) from the fluid chamber 108 to the fluid collection container. In this way, the fluid flow path is provided from the nipple receiving portion 194 to the fluid collection container via the fluid chamber 108 of the valve assembly.

The breast receiving part 192 includes a nipple receiving portion 194 and a breast contacting portion 195 configured to engage with a user's breast. The nipple receiving portion 194 is provided as a substantially cylindrical cup, suitably sized to receive a user's nipple. The breast contacting portion 192 is provided as a substantially spherical dome, suitably sized to contact the skin around a user's nipple when the nipple is placed inside the nipple receiving portion 194. A first end of the nipple receiving portion 194 opens to the centre of the breast contacting portion 192 to thereby receive the nipple. A second end of the nipple receiving portion 194 includes an opening 196 adapted to allow fluid, such as air or expressed milk, to flow out therefrom.

To use the breast pump 190, the user places the breast horn 192 against their breast so that their nipple is received within the nipple receiving portion 194. The nipple is received until the breast contacting portion 192 engages with the skin around the nipple, forming a substantially air-tight seal around the open end of the nipple receiving portion 194.

The user activates the negative pressure source so that a first negative pressure cycle commences as described above. Negative pressure in the fluid chamber 108 provides a pressure difference across the first one-way valve 162 allowing it to open and draw fluid from the nipple receiving portion 194 and applying negative pressure to the user's nipple, stimulating expression of breast milk.

Fluid, including expressed breast milk, is drawn into the valve assembly 100 from the nipple receiving portion 194 by the negative pressure of fluid chamber 108. As the diaphragm 170 reaches its displaced position, negative pressure in the suction chamber 104 is released, allowing the pressure in the suction chamber 104 to return towards atmospheric pressure, and thereby allowing the diaphragm to return to its neutral position.

The initial pressure release in the suction chamber 104 causes the diaphragm 170 to attempt to return towards its undeformed shape. The diaphragm 170 thus attempts to move towards its neutral position and decrease the volume of the fluid chamber 108.

In practice however, only a portion of fluid may be expelled from the fluid chamber 108 through the outlet 122 before the negative pressure in the suction chamber 104 returns to atmospheric pressure.

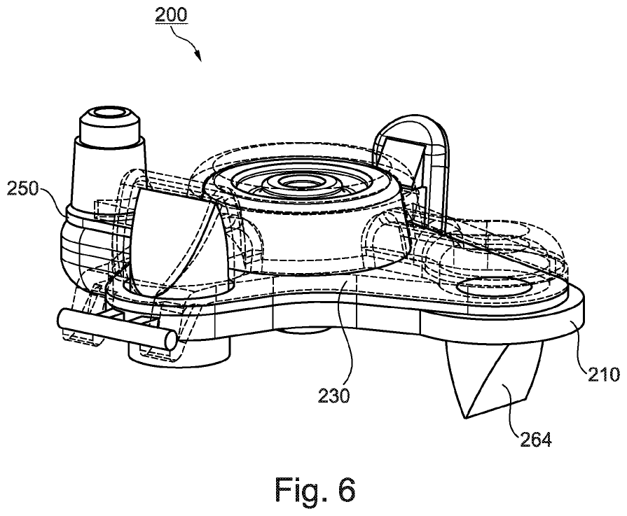

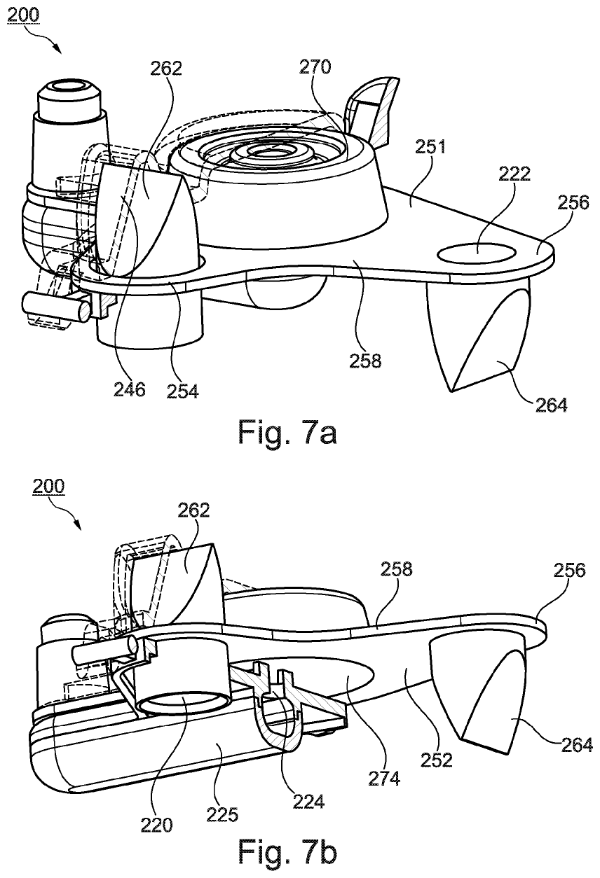

Referring now to FIGS. 6 to 8, there is shown a second known example breast pump valve assembly 200. The example shown in FIGS. 6 to 8 is similar to the first known example shown in FIGS. 1 to 5, other than the first one-way valve 262 and the displacement member 270 are arranged along a first axis, and the second one-way valve 264 and the displacement member 270 are arranged along a second axis arranged perpendicular to the first axis.

In this example, the assembly 200 includes a suction chamber and fluid chamber defined between a first one-way valve 262 and a second one-way valve 264. The valve assembly 200 further includes a displacement member 270, arranged to fluidly separate the suction chamber from the fluid chamber, configured such that application of negative pressure at the start of a first cycle of negative pressure causes the displacement member 270 to move and thereby increase the volume of fluid chamber, drawing liquid into the fluid chamber through the first one-way valve 262.

To complete the first cycle, negative pressure in the suction chamber is released, allowing the displacement member 170 to attempt to return to its undeformed neutral shape.

The valve assembly 200 includes a housing which includes an upper member 230 and a lower member 210, and a support member 250 positioned in between. In contrast to the first example, the lower member 210 is provided as an L-shaped plate having upper and lower surfaces.

The fluid inlet 220 and the vacuum port 224 are arranged along a first axis, and the fluid outlet 222 and the vacuum port are arranged along a second axis arranged perpendicular to the first axis.

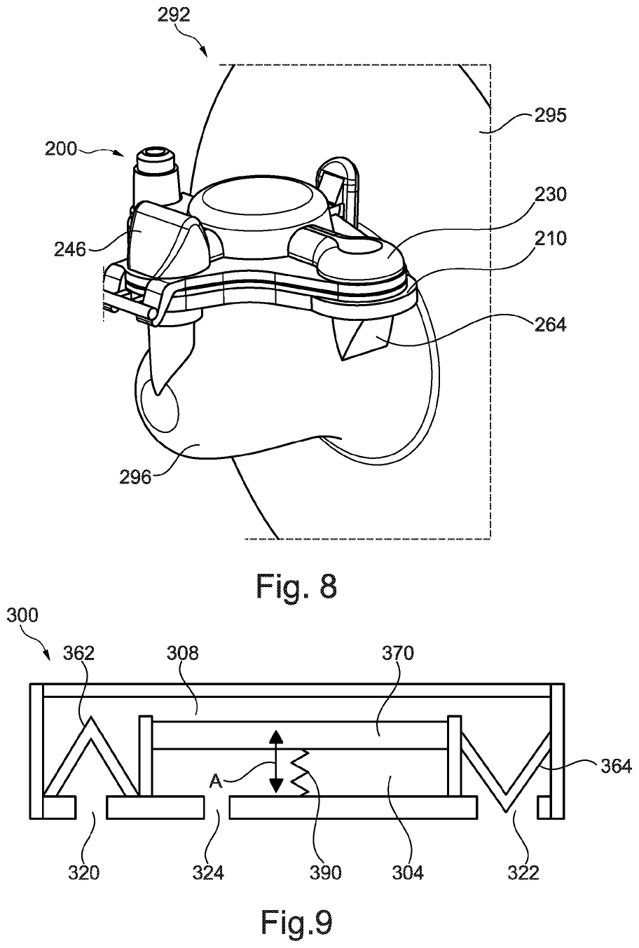

Referring now to FIG. 8, there is shown a known arrangement of the second known valve assembly 200 mounted to a breast horn 292. The breast horn 292 includes a nipple receiving portion 296 and a breast contacting portion 295 shaped and configured to engage with a user's breast in the same manner as the breast horn 192 of FIG. 5. A first end of the nipple receiving portion includes an opening adapted to allow fluid, such as air or expressed milk, to flow out therefrom.

The valve assembly 200 and breast horn may be included in a breast pump, not shown, including a negative pressure source, to be used in the same manner as breast pump 290 described above.

Referring now to FIG. 9, there is shown a third known example valve assembly 300. The example shown is similar to the example described in reference to FIG. 1 other than the displacement member includes a reciprocating member, which is biased to substantially close the fluid chamber 308. The reciprocating member may be in the form of a piston 370 configured to reciprocate within the fluid chamber 308 in the direction of arrows A. A spring 390, or other suitable biasing element, biases the piston to substantially close the fluid chamber 308.

Upon application of negative pressure to the suction chamber 304 via vacuum port or inlet 324, the piston 370 slides to reduce the volume of the suction chamber 304 and increase the volume of the fluid chamber 308. Pressure in the fluid chamber 308, thereby reduces, opening the first one-way valve 362 to draw fluid into the fluid chamber 308 from the fluid inlet 320.

As negative pressure in the suction chamber 304 is released, the suction chamber returns towards atmospheric pressure and the biasing element 390 acts to attempt to move the piston 370 to increase the volume of the suction chamber 304 and decrease the volume of the fluid chamber 308.

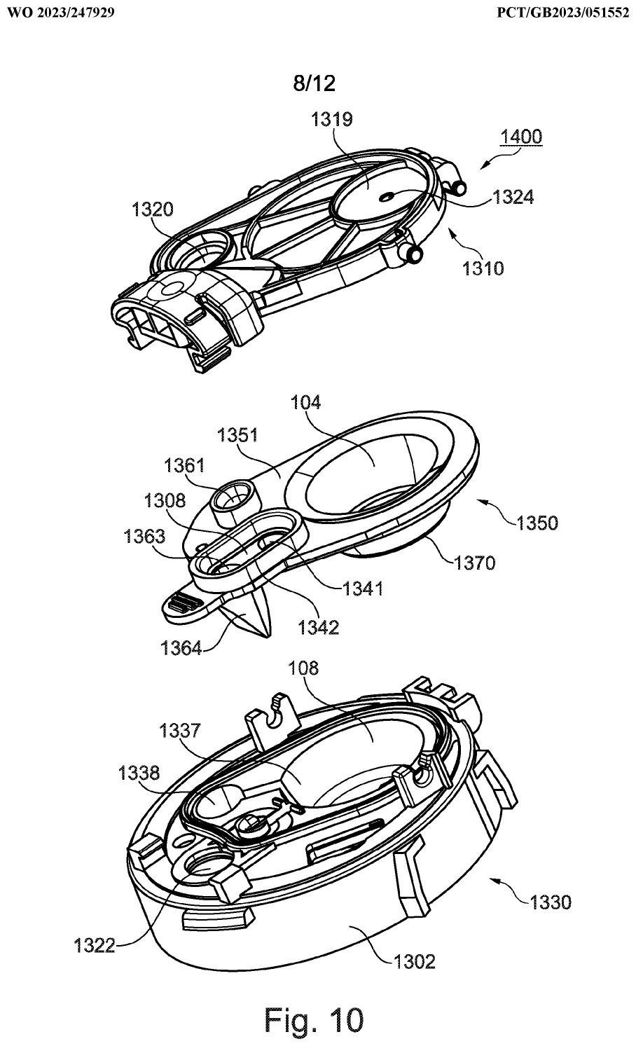

FIGS. 10 to 13 show a fourth known example configuration of the valve assembly 100 of FIG. 1. Where the features are the same as the first example, the reference numbers are the same other than the initial digits are “13”. The example shown in FIGS. 10 to 12 is similar to the first known example shown in FIGS. 1 to 5, but the first one-way valve 1362 is positioned adjacent the second one-way valve 1364. The fluid chamber 108 includes a first fluid flow channel 1339 between the first one-way valve 1262 and the cavity 1337. The fluid camber 108 also includes a second fluid flow channel 1340 between the cavity 1337 and the second one-way valve 1364.

In this example, the valve assembly 1300 includes a housing having a first housing member 1330 and a second housing member 1310. The second housing member is an upper member 1310 and the first housing member is a lower member 1330. The lower member 1330 generally defines the fluid chamber 108. The upper member 1310 generally defines the suction chamber 104. The displacement member 1370 is positioned in between the upper member 1310 and the lower member 1330, thereby fluidly separating the suction chamber 104 and the fluid chamber 108.

In this example, the upper member 1310 includes a first opening, defining the fluid inlet 1320, and a second opening, defining the vacuum port 1324. The vacuum port 1324 may be fluidly coupled to any suitable vacuum source as described herein.

The fluid inlet 1320 may aptly include an inlet port for fluidly coupling the inlet to a breast receiving part of a breast pump.

In this example, a support member 1350 is positioned between the upper member 1310 and the lower member 1330.

The support member 1350 includes a first opening 1361 configured to align with the fluid inlet 1320 in the upper member 1310. The first opening 1361 is provided with the first one-way valve 1362, adapted to selectively allow fluid to pass through the first opening 1361 into the fluid chamber 108.

In this example, the first one-way valve 1362 (see FIG. 13) is a duck bill valve extending from the second surface 1352 of the displacement member 1350. The duck bill valve functions the same as other duck bill valves described herein.

The support member 1350 includes a second opening 1363 configured to align with the fluid outlet 1322 in the lower member 1330. The second opening 1363 is provided with the second one-way valve 1364, adapted to selectively allow fluid to pass through the fluid outlet 1322.

In this example, the second one-way valve 1364 is a duck bill valve extending from the second surface 1352 of the support member 1350. The duck bill valve functions the same as other duck bill valves described herein. In this example, both the first and second one-way valves extend in the same direction.

The support member 1350 further includes the displacement member 1370, which in this example is a flexible diaphragm configured to deform upon application of negative pressure to the suction chamber 104.

Features of the support member 1350 are configured to engage with corresponding features of the upper and lower members 1310, 1330

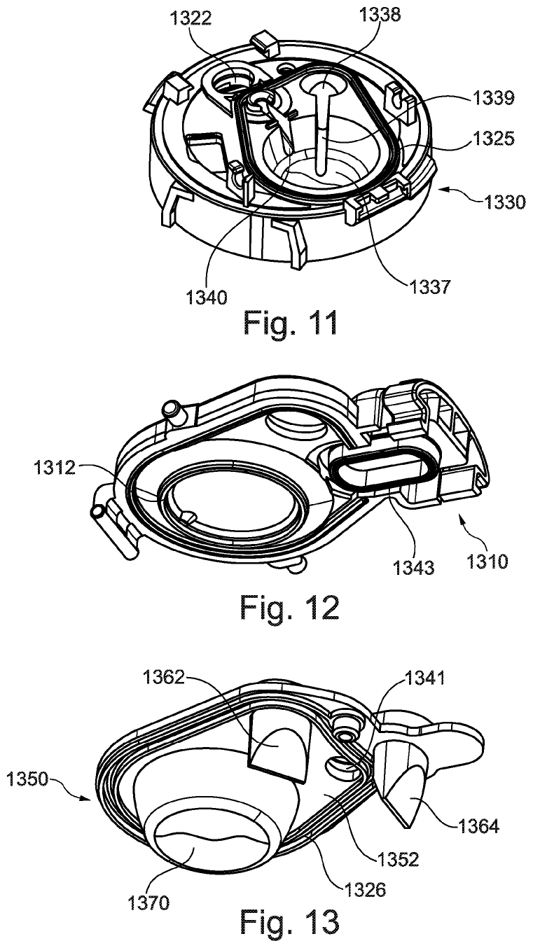

FIG. 11 illustrates a detailed perspective view of the lower member 1330, showing the fluid cavity 1337 and flow channels in further detail. The lower member 1330 includes a first fluid well 1338. The first fluid well 1338 is configured to align with and accommodate at least a portion of the fluid inlet valve 1362 of the support member 1350. As such, in use, fluid enters into the fluid well 1338 from the fluid inlet valve 1362.

The lower member 1330 further includes a first fluid flow channel 1339 extending from the fluid well 1338 to the fluid cavity 1337. In this way, the fluid well 1338 is fluidly coupled to the fluid cavity 1337 via the first fluid flow channel 1339. A second fluid flow channel 1340 extends from the fluid cavity 1337 towards the fluid outlet 1322.

In use, at the start of a first cycle of negative pressure, the diaphragm 1370 is displaced (by a suitable vacuum source) towards the upper member 1310, so that fluid (i.e. breast milk) is pulled into the primary fluid chamber 108 via the first one-way valve 1362, into the fluid well 1338 and along the first fluid flow channel 1339 into the fluid cavity 1337. To complete the first cycle, negative pressure in the suction chamber is released, allowing the diaphragm 1370 to attempt to return to its undeformed neutral shape. The diaphragm 1370 therefore attempts to move back to its neutral position in which a portion of the fluid chamber is substantially closed.

### Breast Pump Valve Assemblies of the Invention

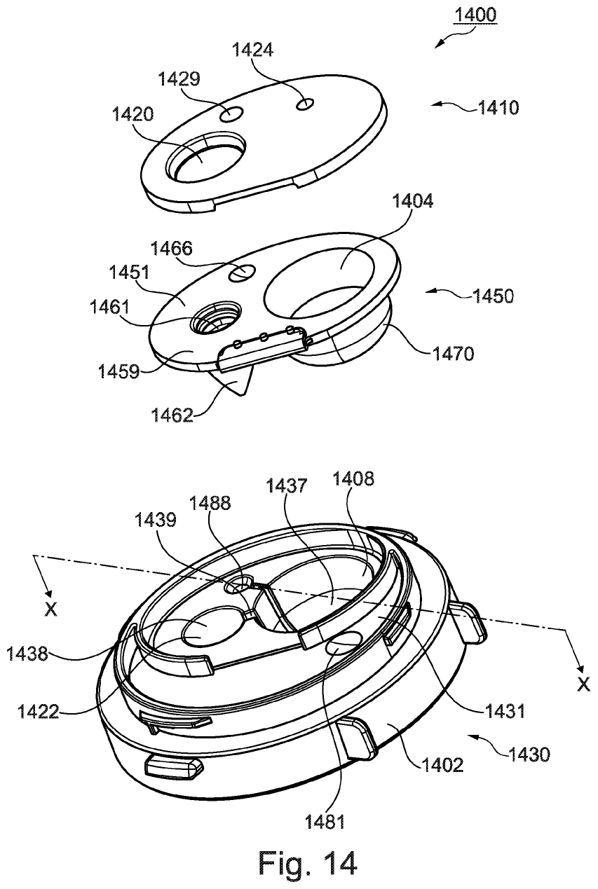

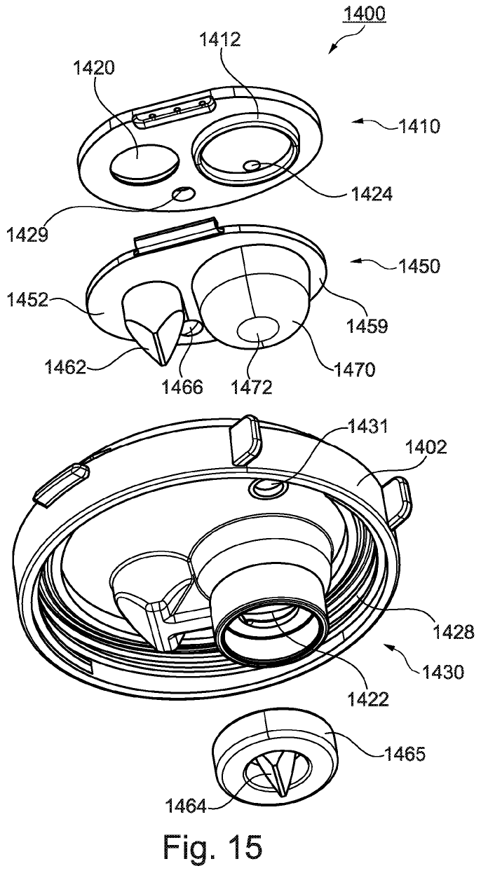

Referring now to FIGS. 14 to 16, there is shown an example configuration of a breast pump valve assembly 1400 according to the present invention. The valve assembly 1400 includes a housing having a first housing member and a second housing member. The first housing member is an upper member 1410 and the second housing member is a lower member 1430.

The upper member 1410 generally defines a first chamber 1404. The first chamber 1404 is essentially a suction chamber as is described herein with reference to FIGS. 1 to 13. In this way, the first chamber 1404 is configured to be fluidly connected to a negative pressure source, such as any negative pressure source described herein.

The lower member 1430 generally defines a second chamber 1408. The second chamber 1408 is essentially a fluid chamber as is described herein with reference to FIGS. 1 to 13. In this way, the second chamber 1408 is defined between a first one-way valve 1462 and a second one-way valve 1464, wherein the first one-way valve 1462 is configured to allow flow of fluid into the second chamber 1408 and the second one-way valve 1464 is configured to allow flow of fluid out of the second chamber 1408.

The displacement member 1470 is positioned between the upper member 1410 and the lower member 1430, thereby fluidly separating the first chamber 1404 and second chamber 1408. That is, the displacement member 1470 ensures the first chamber 1404 is isolated from the second chamber 1408 such that, in use, fluid cannot move between the first chamber 1404 to the second chamber 1408.

The displacement member 1470 is configured such that application of negative pressure to the first chamber 1404 causes a first movement of the displacement member 1470 to thereby increase the volume of the second chamber 1408 and draw fluid into the second chamber 1408 through the first one-way valve 1462.

The displacement member 1470 is further configured such that, when pressure in the first chamber 1404 is equal to or greater than pressure in the second chamber 1408, the pressure in the first chamber 1404 causes a second movement of the displacement member 1470 to thereby decrease the volume of the second chamber 1408.

The breast pump valve assembly 1400 further includes a fluid flow path between the second chamber 1408 and an ambient atmosphere. The fluid flow path includes a selectively openable third valve 1466. Aptly, the ambient atmosphere is the atmosphere surrounding the breast pump.

In this example, the upper member 1410 includes a first opening, defining a fluid inlet 1420, and a second opening, defining a vacuum port 1424. The vacuum port 1424 may be fluidly coupled to any suitable vacuum source as described herein.

The fluid inlet 1420 may aptly include an inlet port for fluidly coupling the fluid inlet 1420 to a breast receiving part of a breast pump.

In this example, the lower member 1430 is configured as a lid for coupling to an opening of a fluid collection container (not shown). Whilst various lid configurations may be possible, in this example the lower member 1430 includes an annular wall 1402 including a threaded radially inner surface 1428. In this way, the lower member 1430 may be directly coupled to the open neck of a fluid collection container, via a threaded interface to form a fluid tight coupling. This configuration allows fluid to pass directly from the fluid outlet 1422 into a fluid collection container, without the need for intermediate fluid pathways or tubing, which may be difficult for a user to clean.

The lower member 1430 includes a third opening, defining a vent port 1481. When the lower member 1430 is coupled to a fluid collection container, the vent port 1481 vents air from the fluid collection container to an ambient atmosphere. In this way, as the expressed breastmilk is collected in the fluid collection container any excess pressure is released through the secondary vent port 1481.

In this example, the lower member 1430 includes a cavity 1437, which defines at least a portion of the second chamber 1408 between the first one-way valve 1462 and second one-way valve 1464. In this example, the cavity 1437 is circular in cross-section. However, it will be appreciated that other cavity shapes may also be suitable.

The lower member 1430 includes a first fluid well 1438. The first fluid well 1438 is configured to align with and accommodate at least a portion of the fluid inlet valve 1462 of the support member 1450. As such, in use, fluid enters into the first fluid well 1438 from the fluid inlet valve 1462.

The lower member 1430 further includes a first fluid flow channel 1439 extending from the first fluid well 1438 to the fluid cavity 1437. In this way, the first fluid well 1438 is fluidly connected to the fluid cavity 1437 via the first fluid flow channel 1439.

A second fluid flow channel extends from the fluid cavity 1437 through the lower member 1430 towards the fluid outlet 1422. In this example, the fluid outlet 1422 is a tube portion extending from the underside of the lower member 1430. A valve element 1465 including a second one-way valve 1464 is mounted to the fluid outlet 1422. The second one-way valve 1464 is a duck bill valve provided on the fluid outlet 1422 of the support member 1450. The duck bill valve functions the same as other duck bill valves described herein. The valve element 1465 is mountable to and demountable from the lower member. This allows for easy cleaning of the valve element.

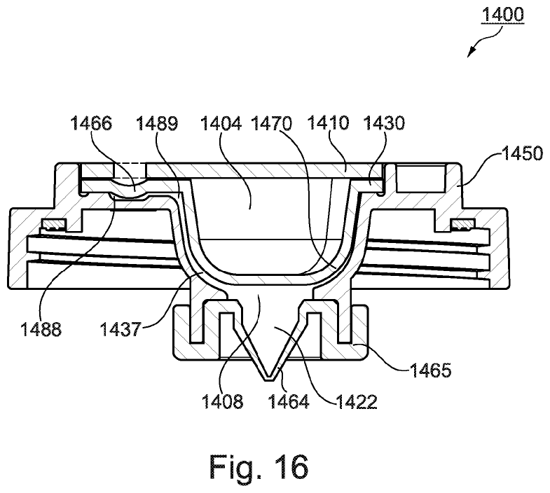

Referring particularly to FIG. 16, there is shown a cross-sectional view of the lower member 1430 along the plane X-X shown in FIG. 14. The lower member 1430 includes a third fluid well 1488. The third fluid well 1488 is configured to align with and accommodate at least a portion of the third valve 1466 of the support member 1450.

The lower member 1430 further includes a third fluid flow channel 1489 extending from the fluid cavity 1437 to the third fluid well 1488. In this way, the third fluid well 1488 is fluidly connected to the fluid cavity 1437 via the third fluid flow channel 1489. The third valve 1466, third fluid well 1488 and the third fluid flow channel 1489 together form a fluid flow path from the second chamber 1408 and the ambient atmosphere.

Aptly, the third fluid flow channel 1489 is a recessed channel. The recessed channel is provided in the wall of the fluid cavity 1437 of the lower member 1430.

Aptly, the third fluid flow channel 1489 is configured to limit or restrict flow of a liquid therethrough. For example, when formed as a recessed channel, the width of the recessed channel may be sufficiently narrow such that flow of a liquid, for example breastmilk, is limited or restricted due to friction between the liquid and the internal walls of the channel. The dimensions may thereby selectively permit air to be directed along the third fluid flow channel.

In this example, the support member 1450 is positioned between the upper member 1410 and the lower member 1430. The support member 1450 includes the displacement member 1470 and the first one-way valve 1462. In this way, the displacement member 1470 and first one-way valve 1462 are integrally formed.

The support member 1450 includes a first surface 1451 facing the upper member 1410 and a second surface 1452 facing the lower member 1430. The support member 1450 includes a first opening 1461 configured to align with the fluid inlet 1420 in the upper member 1410. The first opening 1461 is provided with the first one-way valve 1462, adapted to selectively allow fluid to pass through the first opening 1461 into the second chamber 1408.

In this example, the first one-way valve 1462 is a duck bill valve extending from the second surface 1452 of the displacement member 1450. The duck bill valve functions the same as other duck bill valves described herein.

The support member 1450 includes a third valve 1466 configured to align with the vent port 1429 in the upper member 1410. In the present example, the third valve 1466 is fluidly connected to the second chamber 108 though the third fluid well 1488 and the third fluid flow channel 1489. As such, the third valve 1466 is configured to be selectively openable to allow fluid to pass from the second chamber 108 to the ambient atmosphere. Furthermore, the third valve 1466 is configured to be selectively openable to allow fluid from the ambient atmosphere be drawn into the second chamber 108.

In the example shown, the third valve 1466 is a slit through the support member 1450. The third valve 1466 is configured to selectively open in response to a pressure difference between the first surface 1451 and the second surface 1452 in the vicinity of the third valve 1466. That is, the third valve 1466 is biased closed and is opened by a pressure difference between the opposing surfaces of the third valve 1466. The third valve 1466 is configured to open when the pressure difference between the opposing surface provides a greater pressure on the surface of the third valve 1466 adjacent the first surface 1451 of the support member 1450. Aptly, the third valve 1466 may be configured to open when the pressure difference between the opposing surfaces of the third valve 1466 provides a greater pressure on the surface of the third valve adjacent the second surface 1452 of the support member 1450.

In this example the third valve 1466 includes a slit in a recessed dome that is recessed away from the first surface 1451. In this way, the pressure difference required to open the third valve 1466 to allow fluid to pass from the second chamber 108 to the ambient atmosphere is smaller than the pressure difference required to open the third valve 1466 to allow fluid to pass from the ambient atmosphere to the second chamber 108. However, it will be appreciated that the third valve 1466 may be configured so that the pressure difference required to open the third valve be equal for it to open to allow fluid to pass in either direction. Or, the third valve may be configured so that pressure difference required to open the third valve 1466 to allow fluid to pass from the second chamber 108 to the ambient atmosphere is a greater than the pressure difference required to open the third valve 1466 to allow fluid to pass from the ambient atmosphere to the second chamber 108.

The support member 1450 further includes the displacement member 1470, which in this example is a flexible diaphragm. The displacement member 1470 is configured to deform upon application of negative pressure to the first chamber 104.

The displacement member 1470 is positioned such that it aligns with the cavity 1437 in the lower member 1430 and the vacuum port 1424 in the upper member 1410. In this way, the displacement member 1470 is configured to fluidly separate the first chamber 1404, which is defined between the displacement member 1470 and the upper member 1410, and the second chamber 108, which is defined between the cavity 1437 and the displacement member 1470. The upper member 1410 includes an annular seal ring or abutment ring 1412, configured to provide a fluid tight seal around a rim of the displacement member 1470. In this way, a substantially fluid tight first chamber 104 is provided between the upper member 1410 and the displacement member 1470.

The displacement member 1470 may aptly be shaped to correspond to the cavity 1437 in the lower member 1430. In this way, in the neutral position the displacement member 1470 may abut against an inner surface of the cavity 1437, thereby minimizing the volume of the second chamber 1408 as much as possible and ensuring substantially all fluid can be expelled therefrom.

Aptly, in the neutral position, the displacement member blocks the fluid flow path. That is, in the neutral position, the displacement member 1470 covers the opening to the third fluid flow channel. Fluid cannot flow from the second chamber 1408 to the third valve 1466 or vice versa.

The lower member includes a sealing abutment or seal ring 1425 extending around the fluid cavity 1437, the first fluid well 1438, the third fluid well 1488, and the first and third fluid flow channels 1439, 1489. The sealing abutment or seal ring 1425 is configured to releasably couple the support member 1450 and the upper member 1410 to the lower member 1430 to thereby form a complete valve housing with the support member 1450 positioned in between. The upper member 1410 and the lower member 1430 are each adapted to releasably couple together in order to form the complete housing.

When formed in a complete housing, the second surface of the support member 1450 is held against the lower member 1410 by the upper member 1410 to provide a seal between the second surface 1452 of the support member 1450 and the lower member 1430. Optionally, a cover member (not shown) may be releasably coupled to the lower member 1410 to bear against the upper surface of the upper member 1410 and lock the support member 1450 in its sealed position against the lower member 1430.

In use, negative pressure may be introduced to the first chamber 1404 via the vacuum port 1424, using any suitable negative pressure source, for example a manual or electric pump. The negative pressure source may provide pulses or cycles of negative pressure, as known in the art. The breast pump valve assembly 1400 thereby provides pulses or cycles of negative pressure to the breast receiving part of a breast pump.

A negative pressure cycle includes a first part and a second part.

Within a first part of a negative pressure cycle, application of negative pressure within the first chamber 1404 deforms the displacement member 1470 to thereby decrease the volume of the first chamber 1404. That is, upon application of negative pressure to the first chamber 1404, the volume of the second chamber 1408 is thereby increased by the first movement of the displacement member 1470. The displacement member 1470 moves away from the lower member 1410.

As the volume of the second chamber 1408 increases, the pressure in the second chamber 1408 decreases, thereby creating a pressure differential across the first one-way valve 1462. Due to the pressure differential, the first one-way valve 1462 opens to draw fluid from the fluid inlet 1420 into the second chamber 1408.

As negative pressure increases, the displacement member 1470 further deforms, moving further away from the lower housing 1410. Negative pressure in the first chamber 1404 increases until the first cycle reaches a maximum negative pressure. At the maximum negative pressure, the displacement member 1470 is in a displaced position. In the displaced position, the volume of second chamber 1408 is at a maximum and the volume of the first chamber 1404 is at a minimum.

Within the second part of the negative pressure cycle, negative pressure in the first chamber 1404 is released, such that pressure in the first chamber 1404 returns to ambient pressure. Air from the ambient atmosphere is vented into the first chamber 1404 through the vacuum port 1424, equalising the pressure between the first chamber and the ambient atmosphere. Thus, the pressure in the first chamber 1404 is greater than the pressure in the second chamber 1408. The displacement member 1470 thereby attempts to return to its undeformed neutral shape, increasing the pressure upon fluid that has been drawn into the second chamber 1408.

Thus, releasing negative pressure from the first chamber 1404 initiates the second movement of the displacement member 1470 from its displaced position towards its neutral position. The displacement member 1470 therefore attempts to move back to its neutral position in which a portion of the second chamber 1408 is substantially closed.

In the absence of a negative pressure in the first chamber 1404, the displacement member 1470 is biased towards the neutral position. The second movement thereby increases the pressure applied to the fluid that has been drawn into the second chamber 1408. Typically, the release of negative pressure causes a sudden pressure increase to be applied to the fluid that has been drawn into the second chamber 1408.

The third valve 1466 is configured to be selectively opened in response to the second movement of the displacement member 1470. That is, the third valve opens in response to the increase in pressure applied to the fluid drawn into the second chamber 1408. Fluid from the second chamber 1408 is thus directed through the third valve 1466 via the third fluid flow channel 1489 and the third fluid well 1488. In this way, the initial pressure applied to the fluid in the second chamber 1408 is released by fluid passing through the third valve 1466. The displacement member 1470 thereby moves towards the neutral position with reduced resistance from the fluid in the second chamber 1408.

In this example, in the neutral position, the lower surface of the displacement member 1470 abuts a portion of the wall of the cavity 1437. The fluid outlet 1422 of the lower member includes a longitudinal axis. The base 1472 of the displacement member 1470 is aligned with the longitudinal axis of the fluid outlet 1422. Thus, a movement of the displacement member 1470 moves the base 1472 in the direction of the longitudinal axis.

When moving from the displaced position to the neutral position, the second chamber 1408 contracts to its minimal volume. The base 1472 of the displacement member 1470 thereby moves in the direction of the longitudinal axis and towards the fluid outlet 1422. In this way, fluid is directed to the second one-way valve 1464 with reduced friction. Fluid is thereby directed more easily to the second one-way 1464 and displaced to the collection container in an efficient manner.

The initial release of fluid through the third valve 1466 thereby ensures substantially all fluid can be expelled from the second chamber 1408. The second movement of the displacement member 1470 encounters reduced resistance and thereby directs substantially all fluid from the second chamber 1408.

Typically, the fluid directed through the third valve 1466 is air. In particular, the third fluid flow channel 1489 has dimensions to limit or restrict flow of liquid therethrough, as described above. In this way, in response to the second movement, air is directed initially through the third valve 1466. The resistance to the second movement is reduced allowing the displacement member 1470 to continue moving toward the neutral position and direct substantially all remaining fluid, typically breastmilk, through the second one-way valve 1464 and into the fluid collection container.

In the example shown in FIGS. 14 to 16, the upper and lower members 1410, 1430 are formed from a rigid material, for example a plastic such as polypropylene. Polypropylene (PP) which may be particularly advantageous since it is food contact safe, suitable for sterilisation and is inert to cleaning chemicals. In some examples, the upper and lower member may be formed from a filled PP, for example PP plus CaCO₃, to provide greater strength.

The support member 1450, including the displacement member 1470 and first and second one-way valves 1462, 1464, is formed from a fluid impermeable flexible material. The material is aptly resiliently deformable. For example, suitable materials may include an elastomeric polymer such as silicone rubber. The material may aptly have a Shore A hardness of 30 to 70 Shore A, or aptly 50 Shore A.

Various modifications to the detailed arrangements as described above are possible. For example, the support member may be omitted and the displacement member may be provided as a separate part to the first and/or second one-way valves. In another example, the support member may include the first and second one-way valves and the displacement member may be a separate part. The displacement member may be mounted to the lower member to form the first chamber and the second chamber may be formed by releasably coupling the upper and lower members.

Aptly, in use, fluid may be drawn into the second chamber 1408 through the third valve 1466. The first portion of the negative pressure cycle creates suction in the second chamber 1408 which, in turn provides a pressure difference across the third valve 1466. The pressure from the ambient atmosphere is greater than the pressure within the third fluid well 1488. The third valve 1466 opens to allow fluid, typically air, to be drawn along the third fluid flow channel 1489 and into the second chamber 1408.

Additionally, or alternatively, one or more of the one-way valves may be separate components, mounted to or within the corresponding fluid inlet or outlet. Although duckbill valves are described, other types of valve may be suitably employed, for example dome valves or slit membrane valves.

Although in the examples described above, the fluid inlet, fluid outlet and vacuum port are positioned in the lower member, one or more of the fluid inlet, fluid outlet and vacuum port may be positioned in other suitable locations. So long as the variable volume second chamber is positioned between the first and second one-way valves, the first and second one-way valves and corresponding inlet and outlet may be positioned in any suitable location in the valve assembly.

Similarly, the vacuum port may be positioned in any location so long as it is in fluid communication with the first chamber.

The displacement member may be any member suitable for varying the volume of the second chamber. For example, the displacement member may include a reciprocating member, which is biased to substantially close the second chamber. The reciprocating member may be in the form of a piston configured to reciprocate within the second chamber. A spring, or other suitable biasing element, biases the piston to substantially close the second chamber.

Upon application of negative pressure to the first chamber via a vacuum port or inlet, the piston slides to reduce the volume of the first chamber and increase the volume of the second chamber. Pressure in the second chamber, thereby reduces, opening the first one-way valve to draw fluid into the second chamber from the fluid inlet.

As negative pressure in the first chamber is released, the first chamber returns towards atmospheric pressure and the biasing element acts to move the piston to increase the volume of the first chamber and decrease the volume of the second chamber. In doing so, the first one-way valve 362 closes to prevent back flow of fluid and the third valve opens to allow fluid to exit the second chamber. Subsequently, the second one-way valve opens to allow fluid to exit the second chamber through the outlet. The cycle may then be repeated in the same way as described above with reference to FIGS. 14 to 16.

The reciprocating motion of the piston may alternatively be achieved through manual actuation of a piston rod, for example, mitigating the need for the first chamber, biasing element and negative pressure source.

Any suitable pump may be used with the breast pump valve assemblies described above including, but not limited to, negative pressure sources such piezoelectric pump, a diaphragm pump, a reciprocating pump, or a peristaltic pump. Alternatively, manually actuated pumps, such as membrane or piston pumps may provide a negative pressure.

Further alternatively, the negative pressure source may be replaced by a device which is actuated either manually or by an electric motor and thereby move the displacement member in order to vary the volume of the second chamber. Negative pressure sources as well as actuated devices may be provided in a unit integral with the breast pump valve assembly, or may be provided remote from the breast pump assembly and operably connected via a suitable conduit.

The valve assemblies described herein provide breast pump that advantageously does not release the negative pressure between the breast pump and the user's breast between negative pressure cycles or pulses. The user is thus able to use a breast pump without having to apply pressure to the breast, thereby reducing tiredness and discomfort. Furthermore, there is a reduced risk of breastmilk leaking from the breast pump during expression.

The valve assemblies described herein also provide the advantage that a negative pressure at the nipple is generated using reduced movement of the displacement member. Consequently, the displacement member need only move a small distance when moving from a neutral position to a displaced position, resulting in a valve assembly and a breast pump with reduced size and weight.

The valve assemblies described herein advantageously provide suction with increased efficiency because the suction applied by the breast pump is not repeatedly released and regenerated with every suction cycle. Stimulated expression of breastmilk is provided efficiently and in a continuous manner without interruption.

With the arrangement of the invention, operational performance of the pump assembly is improved. Fluid flow of breastmilk within the fluid chamber is improved, and therefore passes directly to the collection chamber.

The breast pump described herein provides improved collection of breastmilk. The breast pump is able to expel or displace into a collecting container a substantial portion, or substantially all, of the breastmilk drawn into the breast pump valve assembly. More particularly, the breast pump is able to expel or displace into a collecting container a substantial portion, or substantially all, of the breastmilk drawn into the breast pump valve assembly during each suction cycle. Any fluid drawn into the valve assembly is actively pushed out of the valve assembly outlet to the fluid collection container.

Leakage of breastmilk from the assembly is also avoided. Providing a two-part housing and support member as described above allows for simplified assembly and disassembly of the valve assembly allowing the user to easily clean the valve assembly.

The valve assembly described herein may be compatible with a range of different negative pressure sources or actuating devices. Furthermore, the valve assembly may be used in a variety of breast pump configurations including manual pumps and electric pumps and form part of a wearable or non-wearable breast pump assembly.

It will be clear to a person skilled in the art that features described in relation to any of the embodiments described above can be applicable interchangeably between the different embodiments. The embodiments described above are examples to illustrate various features of the invention.

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference.

1. A breast pump valve assembly comprising:
a first chamber for fluid connection to a negative pressure source;
a second chamber defined between a first one-way valve and a second one-way valve, wherein the first one-way valve is configured to allow flow of fluid into the second chamber and the second one-way valve is configured to allow flow of fluid out of the second chamber,
a fluid flow path between the second chamber and an ambient atmosphere, the fluid flow path comprising a selectively openable third valve; and
a displacement member fluidly separating the first chamber and the second chamber, wherein the displacement member is configured such that application of negative pressure to the first chamber causes a first movement of the displacement member to thereby increase the volume of the second chamber and draw fluid into the second chamber through the first one-way valve,
wherein the displacement member is further configured such that, when pressure in the first chamber is equal to or greater than pressure in the second chamber, the pressure in the first chamber causes a second movement of the displacement member to thereby decrease the volume of the second chamber; and
wherein the third valve is configured to be selectively opened in response to the second movement of the displacement member, such that the second movement of the displacement member directs fluid from the second chamber through the third valve.

2. A breast pump valve assembly according to claim 1, wherein the displacement member is configured such that the second movement of the displacement member further directs fluid from the second chamber through the second one-way valve.

3. A breast pump valve assembly according to claim 1, wherein the third valve is further configured to be selectively openable in response to the first movement of the displacement member, thereby drawing fluid into the second chamber through the third valve.

4. A breast pump valve assembly according to claim 1, wherein the displacement member is configured to move between a neutral position when the first chamber is at atmospheric pressure, and a displaced position when the first chamber is under negative pressure.

5. A breast pump valve assembly according to claim 4, wherein in the neutral position, the displacement member substantially closes at least a portion of the second chamber.

6. A breast pump valve assembly according to claim 5, wherein in the neutral position, the displacement member blocks the fluid flow path.

7. A breast pump valve assembly according to claim 1, wherein the displacement member is integrally formed with one or more of: the first one-way valve, the second one-way valve, or the third valve.

8. A breast pump valve assembly according to claim 1, wherein the displacement member comprises a flexible diaphragm configured to deform upon application of negative pressure to the first chamber.

9. A breast pump valve assembly according to claim 1, wherein at least one of the first and second one-way valves is a duck bill valve.

10. A breast pump valve assembly according to claim 1, wherein the third valve is a slit valve, and preferably a domed cross-cut slit valve.

11. A breast pump valve assembly according to claim 1, further comprising a housing comprising a first housing member defining the second chamber, and a second housing member defining the first chamber and, preferably, wherein the displacement member is positioned between the first housing member and the second housing member.

12. A breast pump valve assembly according to claim 1, further comprising a fluid inlet, wherein the first one-way valve is configured to allow flow of fluid from the fluid inlet to the second chamber and, preferably, further comprising an inlet port for fluidly coupling the inlet to a breast receiving part of a breast pump.

13. A breast pump valve assembly according to claim 1, further comprising a fluid outlet, wherein the second one-way valve is configured to allow flow of fluid from the second chamber to the outlet and, preferably, further comprising an outlet port for fluidly coupling the outlet to a fluid collection container.

14. A breast pump valve assembly according to claim 1, further configured such that, in response to the second movement of the displacement member, the fluid flow path directs air from the second chamber through the third valve.

15. A breast pump valve assembly according to claim 14, wherein the fluid flow path is a recessed channel.

16. A breast pump valve assembly comprising:
a variable volume second chamber defined between a first one-way valve and a second one-way valve, wherein the first one-way valve is configured to allow flow of fluid into the second chamber and the second one-way valve is configured to allow flow of fluid out of the second chamber,
a third valve is configured to provide a selectively openable fluid flow path between the second chamber and an ambient atmosphere; and
a displacement member configured for varying the volume of the second chamber, wherein the displacement member is configured such that a first movement of the displacement member to increase the volume of the second chamber thereby draws fluid into the second chamber through the first one-way valve;
wherein the third valve is configured to be selectively opened in response to a second movement of the displacement member, thereby directing fluid from the chamber through the third valve.

17. A breast pump valve assembly comprising according to claim 16, wherein the displacement member is further configured such that the second movement of the displacement member decreases the volume of the second chamber.

18. A breast pump valve assembly according to claim 16, further comprising a first chamber, wherein the displacement member fluidly separates the first chamber and the second chamber.

19. A breast pump valve assembly according to claim 18, further comprising a vacuum port for fluidly connecting the first chamber to a negative pressure source.

20. A breast pump valve assembly according to claim 19, wherein the displacement member is configured such that application of negative pressure to the first chamber causes the first movement of the displacement member to thereby increase the volume of the second chamber and draw fluid through the first one-way valve into the second chamber.

21. A breast pump valve assembly according to claim 16, wherein the displacement member is further configured such that, when pressure in the first chamber is equal to or greater than pressure in the second chamber, the pressure in the first chamber causes the second movement of the displacement member.

22. A breast pump valve assembly according to claim 16, further comprising an actuator configured to operably move the displacement member to thereby vary the volume of the second chamber.

23. A breast pump comprising:
a first chamber and a vacuum port for fluidly connecting the first chamber to a negative pressure source;
a breast receiving part, a fluid collection container, and a fluid flow path extending between the breast receiving part and the fluid collection container;
a second chamber defined between a first one-way valve and a second one-way valve in the fluid flow path, wherein the first one-way valve is configured to allow flow of fluid from the breast receiving part into the second chamber, and the second one-way valve is configured to allow flow of fluid from the second chamber to the fluid collection container,
a fluid flow path between the second chamber and an ambient atmosphere, the fluid flow path comprising a selectively openable third valve; and
a displacement member fluidly separating the first chamber and the second chamber, wherein the displacement member is configured such that application of negative pressure to the first chamber causes a first movement of the displacement member to thereby increase the volume of the second chamber and draw fluid from the breast receiving part through the first one-way valve into the second chamber wherein the displacement member is configured such that, increase of pressure in the first chamber towards atmospheric pressure causes a second movement of the displacement member to thereby decrease the volume of the second chamber; and
wherein the third valve is configured to be selectively openable in response to the second movement of the displacement member, thereby directing fluid from the second chamber through the third valve.

24. A breast pump according to claim 23, wherein the displacement member is configured such that the second movement of the displacement member further directs fluid from the second chamber through the second one-way valve to the fluid collection container.

25. A breast pump according to claim 23, further comprising a negative pressure source fluidly connected to the vacuum port.
